(19) United States
(12) Patent Application Publication
(10) Pub. No.: US 2023/0119836 A1
(43) Pub. Date: Apr. 20, 2023

(54) CHINESE HERBAL MEDICINE COMPOSITION FOR RELIEVING HEARING LOSS AND PREPARATION METHOD AND USE THEREOF

(71) Applicant: Infinitus (China) Company Ltd., Jiangmen (CN)

(72) Inventors: Yaqian GAO, Jiangmen (CN); Jian TANG, Jiangmen (CN); Yiting YANG, Jiangmen (CN); Xiaochun XIAO, Jiangmen (CN); Zhiyun DU, Jiangmen (CN); Shijing MA, Jiangmen (CN); Li LIN, Jiangmen (CN)

(73) Assignee: Infinitus (China) Company Ltd., Jiangmen (CN)

(21) Appl. No.: 17/569,958

(22) Filed: Jan. 6, 2022

(30) Foreign Application Priority Data
Oct. 18, 2021 (CN) 202111208294.3

(51) Int. Cl. A61K 36/9066; A61P 27/16; A61K 36/8984; A61K 36/67

(52) U.S. Cl. CPC A61K 36/9066; A61P 27/16; A61K 36/8984; A61K 36/67; A61K 2236/333

(57) ABSTRACT

The invention discloses a Chinese herbal medicine composition for relieving hearing loss and a preparation method and use thereof. The Chinese herbal medicine composition is prepared from at least two of the medicinal and edible raw materials of rhizoma curcumae longae, Dendrobium nobile and fructus Piperis longi, can effectively reduce the content of NO secreted by cells, reduces cell inflammation, and has a good effect of relieving hearing loss induced by LPS. The Chinese herbal medicine composition is not a simple combination of traditional Chinese medicines and has a synergistic effect. The Chinese herbal medicine composition can significantly reduce ABR value, MDA, and glutamic acid of a patient with hearing loss, significantly relieves hearing loss, and can be developed into functional foods and medicines for relieving hearing loss as an active ingredient.

### CROSS-REFERENCE TO RELATED APPLICATION

This application claims the benefit of priority to Chinese Patent Application No. 202111208294.3, filed Oct. 18, 2021, the entire disclosure of which is incorporated herein by reference.

### TECHNICAL FIELD

The invention belongs the technical field of foods and medicines, and particularly relates to a Chinese herbal medicine composition for relieving hearing loss and a preparation method and use thereof.

### BACKGROUND

Hearing loss is a pathological change in reduction of hearing sensitivity of human ears, including mild-to-severe hearing loss and deafness. Studies show that the hearing loss has become a common disease affecting the population all over the world. Factors inducing the hearing loss even deafness include heredity, environmental noise pollution, aging, age as well as chronic diseases such as diabetes, hypertension, hyperlipidemia, otitis media and the like, which seriously affect health and life of people. Currently, main clinical methods for treating the hearing loss and deafness include implanting an artificial cochlea, wearing a hearing aid and taking medicines such as midecamycin tablets, antideaf otic pills, magnet and cinnabar pills and Yufeng ningxin tablets. The drug therapy has a certain effect on improving a hearing function. However, the medicine taking for a long time may have certain side effects on human body. The wearing of the hearing aid treats symptoms rather than root causes. The implanting of the artificial cochlea is expensive and can damage an original cochlear tissue, such that a patient permanently loses the chance of the drug therapy. Nevertheless, up to now, no functional foods have been marketed for relieving hearing loss or even deafness. At present, a series of medicinal and edible Chinese herbal medicines published by the National Health Commission of the People's Republic of China can be developed into functional foods, serve as medicinal diets to relieve body diseases or sub-health states, can also reduce side effects brought by the medicines and contribute to recovery of body functions. Therefore, it is of great significance in developing functional foods and medicines for relieving hearing loss by using natural Chinese herbal medicines as main raw materials.

In the prior art, Radix puerariae, Radix salviae miltiorrhizae, Cornus officinalis and other Chinese herbal medicines are mainly used to develop a combination of Chinese herbal medicines, which is mainly used for treating people with deafness and other diseases. For example, Patent CN1173371A discloses a deafness-treating traditional Chinese medicine prepared from 19 traditional Chinese medicines composed of Radix puerariae, Radix salviae miltiorrhizae, Radix bupleuri, rhizoma acori graminei, Radix rehmanniae preparata, honeysuckle, wild chrysanthemum, etc.; this deafness-treating traditional Chinese medicine can open orifices, promote blood circulation and collaterals, nourish the kidneys and improving hearing, and mainly treat sudden deafness, neurological deafness, senile deafness, and drug-toxic deafness; and this deafness-treating traditional Chinese medicine does not have a high effective cure rate (a cure rate of 38.2 to 67.2%) according to clinical verification of 848 cases. In Patent CN106620011A, honeysuckle, fructus trichosanthis, Radix angelicae sinensis, rhizoma corydalis, rhizoma chuanxiong, ginseng, trogopterus dung, borneol, musk or synthetic musk, magnet, fructus lycii, dandelion and Cornus officinalis are prepared into an eardrop preparation which can promote regeneration and repair of damaged and dead sensory mother cells and conduction nerves, and thus restores a hearing function. However, in the prior art, a medicine in a form of a combination of traditional Chinese medicines is obtained by a deafness evaluation model or a clinical evaluation and mainly for people suffering from deafness, and relieves hearing loss caused by environmental noise, body aging and inflammation in daily life. There are few reports on the medicinal and edible functional foods for people who are not in an illness state. It is found in the previous studies that rhizoma curcumae longae, Dendrobium officinale and fructus Piperis longi have good effects on human sub-health and diseases such as aging, inflammation, fatigue, hyperglycemia, hyperlipidemia, etc., but there are few studies on hearing loss. The above Chinese herbal medicines are all medicinal and edible raw materials. In addition, most of the Chinese herbal medicines used in foods or technologies for relieving hearing loss and deafness contain active ingredients such as terpenoids, flavonoids, alkaloids, and phenols. Since the extract powder finished products obtained by extraction, drying, etc. have unpleasant feelings of bitter and pungent taste, a large amount of sweeteners, flavoring agents and other auxiliary materials are needed to improve the taste, which may affect consumer experience and efficacy.

### SUMMARY

The purpose of the first aspect of the invention is to provide use of rhizoma curcumae longae in preparing a product.

The purpose of the second aspect of the invention is to provide a Chinese herbal medicine composition.

The purpose of the third aspect of the invention is to provide a preparation method of a Chinese herbal medicine composition.

The purpose of the fourth aspect of the invention is to provide use of the Chinese herbal medicine composition of the second aspect in preparing a product.

The purpose of the fifth aspect of the invention is to provide a product.

The purpose of the sixth aspect of the invention is to provide use of the Chinese herbal composition of the second aspect and/or the product of the fifth aspect in treating hearing loss.

In order to realize the above purposes, a technical solution used in the present invention is as follows:

The first aspect of the invention provides use of rhizoma curcumae longae in preparing a product.

The product has any one of functions from (1) to (6):

(1) preventing and/or treating hearing loss;

(2) reducing an auditory brainstem response (ABR) value;

(3) reducing the content of malondialdehyde;

(4) reducing the content of glutamic acid;

(5) reducing the content of IL-4; and

(6) inhibiting production of inflammatory factors.

Preferably, the inflammatory factors may include TNF-α and NO; and preferably NO.

Preferably, the inflammatory factors may be induced by lipopolysaccharide.

Preferably, the type of the product may include a food and a medicine.

Preferably, the food may be a functional food.

Preferably, the type of the food may include a beverage, a biscuit, a candy and a pastry.

Preferably, the beverage may be a solid beverage.

Preferably, the medicine may have a dosage form including such as a capsule, a tablet, a granule, a mixture, a decoction, a pill, an electuary, a dripping pill, a liniment, a tincture and a wine.

Preferably, the product may further include a pharmaceutically acceptable auxiliary material or a food-acceptable auxiliary material.

Preferably, the hearing loss may include aging hearing loss, noise-induced hearing loss, and inflammatory hearing loss.

Preferably, the rhizoma curcumae longae may be a rhizoma curcumae longae extract.

Preferably, the rhizoma curcumae longae extract may be prepared by conducting water extraction and/or alcohol extraction of rhizoma curcumae longae as a raw material to obtain a rhizoma curcumae longae extract.

Preferably, the preparation method may include the following steps: 1) mixing raw materials with water and conducting an extraction and a solid-liquid separation to obtain a filtrate A and a medicine residue; 2) mixing the medicine residue obtained in step 1) with ethanol, and conducting an extraction and a solid-liquid separation to obtain a filtrate B; and 3) mixing the filtrate A and the filtrate B to obtain the Chinese herbal medicine composition.

Preferably, the raw materials in step 1) may be crushed and sieved through a 60-mesh sieve before mixing.

Preferably, a mass ratio of the raw materials to water in step 1) may be 1:(10 to 20).

Preferably, the extraction in step 1) may be a subcritical extraction, a decoction extraction, an ultrasonic extraction, a reflux extraction or a percolation extraction; preferably a subcritical extraction.

Preferably, the raw materials in step 1) may be steamed with a water vapor before mixing.

Preferably, the extraction in step 1) may be conducted at 160 to 200° C. and 2 to 4 MPa for 30 to 90 min; preferably at 160 to 180° C. and 3 to 4 MPa for 30 to 60 min.

Preferably, the extraction in step 1) may be conducted for 1 to 3 times.

Preferably, the mass-volume ratio (g/mL) of the medicine residue to the ethanol in step 2) may be 1:(15 to 35); preferably 1:(15 to 25).

Preferably, the ethanol may have a concentration of 40 to 70 v/v %; preferably 40 to 60 v/v %.

Preferably, the extraction in step 2) may be a subcritical extraction, a decoction extraction, an ultrasonic extraction, a reflux extraction or a percolation extraction; preferably, a decoction extraction.

Preferably, the extraction in step 2) may be conducted at 60 to 90° C. for 1 to 3 h.

Preferably, the extraction in step 2) may be conducted for 1 to 3 times.

Preferably, the preparation method may further include the following steps: conducting primary concentration, homogenization, secondary concentration and drying of a mixture of the filtrate A and the filtrate B.

Preferably, the primary concentration may be conducted at 60 to 75° C. and −0.3 to −0.5 MPa (a vacuum degree), and the resulted volume may be ⅓ to ⅙ of the volume of the mixture after concentration.

Preferably, the homogenization may be conducted at 80 to 120 MPa and 30 to 40° C. for 0.3 to 1 h.

Preferably, the homogenization may be conducted for 4 to 6 times.

Preferably, the secondary concentration may be conducted at 60 to 75° C. and −0.3 to −0.5 MPa (a vacuum degree), and the resulted density may be a relative density of 1.2 to 1.5 after concentration.

Preferably, the drying may include at least one of spray-drying, freeze-drying, vacuum-drying and air-blast drying.

The second aspect of the invention provides a Chinese herbal medicine composition and the Chinese herbal medicine composition is prepared from the raw materials comprising any one of (7) to (10):

(7) 35-85 parts by weight of rhizoma curcumae longae and 35-85 parts by weight of fructus Piperis longi;

(8) 35-85 parts by weight of rhizoma curcumae longae and 25-65 parts by weight of Dendrobium nobile;

(9) 35-85 parts by weight of rhizoma curcumae longae, 25-65 parts by weight of Dendrobium nobile and 35-85 parts by weight of fructus Piperis longi; and

(10) 25-65 parts by weight of Dendrobium nobile and 35-85 parts by weight of fructus Piperis longi.

Preferably, the Chinese herbal medicine composition may be prepared from the raw materials comprising any one of (a7) to (a10):

(a7) 40-80 parts by weight of rhizoma curcumae longae and 40-80 parts by weight of fructus Piperis longi;

(a8) 40-80 parts by weight of rhizoma curcumae longae and 30-60 parts by weight of Dendrobium nobile;

(a9) 40-80 parts by weight of rhizoma curcumae longae, 30-60 parts by weight of Dendrobium nobile and 40-80 parts by weight of fructus Piperis longi; and (a10) 30-60 parts by weight of Dendrobium nobile and 40-80 parts by weight of fructus Piperis longi.

Preferably, the Chinese herbal medicine composition may be prepared from the raw materials comprising any one of (b7) to (b10):

(b7) 60-80 parts by weight of rhizoma curcumae longae and 50-80 parts by weight of fructus Piperis longi;

(b8) 60-80 parts by weight of rhizoma curcumae longae and 50-60 parts by weight of Dendrobium nobile;

(b9) 60-80 parts by weight of rhizoma curcumae longae, 50-60 parts by weight of Dendrobium nobile and 50-80 parts by weight of fructus Piperis longi; and (b10) 50-60 parts by weight of Dendrobium nobile and 50-80 parts by weight of fructus Piperis longi.

Preferably, the Chinese herbal medicine composition may be prepared from the raw materials comprising any one of (11) to (15):

(11) 35-85 parts by weight of rhizoma curcumae longae, 35-85 parts by weight of fructus Piperis longi, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle;

(12) 35-85 parts by weight of rhizoma curcumae longae, 25-65 parts by weight of Dendrobium nobile, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle;

(13) 35-85 parts by weight of rhizoma curcumae longae, 25-65 parts by weight of Dendrobium nobile, 35-85 parts by weight of fructus Piperis longi, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle;

(14) 25-65 parts by weight of Dendrobium nobile, 35-85 parts by weight of fructus Piperis longi, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle; and

(15) 35-85 parts by weight of rhizoma curcumae longae, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle.

Preferably, the Chinese herbal medicine composition may be prepared from the raw materials comprising any one of (a11) to (a15):

(a11) 40-80 parts by weight of rhizoma curcumae longae, 40-80 parts by weight of fructus Piperis longi, 20-60 parts by weight of ginseng, 20-60 parts by weight of mulberry, 10-40 parts by weight of Radix puerariae, 10-40 parts by weight of Cornus officinalis, 10-40 parts by weight of Semen ziziphi spinosae, 10-40 parts by weight of Radix astragali, 10-40 parts by weight of peach kernel, 5-20 parts by weight of licorice, and 5-20 parts by weight of honeysuckle;

(a12) 40-80 parts by weight of rhizoma curcumae longae, 30-60 parts by weight of Dendrobium nobile, 20-60 parts by weight of ginseng, 20-60 parts by weight of mulberry, 10-40 parts by weight of Radix puerariae, 10-40 parts by weight of Cornus officinalis, 10-40 parts by weight of Semen ziziphi spinosae, 10-40 parts by weight of Radix astragali, 10-40 parts by weight of peach kernel, 5-20 parts by weight of licorice, and 5-20 parts by weight of honeysuckle;

(a13) 40-80 parts by weight of rhizoma curcumae longae, 30-60 parts by weight of Dendrobium nobile, 40-80 parts by weight of fructus Piperis longi, 20-60 parts by weight of ginseng, 20-60 parts by weight of mulberry, 10-40 parts by weight of Radix puerariae, 10-40 parts by weight of Cornus officinalis, 10-40 parts by weight of Semen ziziphi spinosae, 10-40 parts by weight of Radix astragali, 10-40 parts by weight of peach kernel, 5-20 parts by weight of licorice, and 5-20 parts by weight of honeysuckle;

(a14) 30-60 parts by weight of Dendrobium nobile, 40-80 parts by weight of fructus Piperis longi, 20-60 parts by weight of ginseng, 20-60 parts by weight of mulberry, 10-40 parts by weight of Radix puerariae, 10-40 parts by weight of Cornus officinalis, 10-40 parts by weight of Semen ziziphi spinosae, 10-40 parts by weight of Radix astragali, 10-40 parts by weight of peach kernel, 5-20 parts by weight of licorice, and 5-20 parts by weight of honeysuckle; and

(a15) 40-80 parts by weight of rhizoma curcumae longae, 20-60 parts by weight of ginseng, 20-60 parts by weight of mulberry, 10-40 parts by weight of Radix puerariae, 10-40 parts by weight of Cornus officinalis, 10-40 parts by weight of Semen ziziphi spinosae, 10-40 parts by weight of Radix astragali, 10-40 parts by weight of peach kernel, 5-20 parts by weight of licorice, and 5-20 parts by weight of honeysuckle.

Preferably, the Chinese herbal medicine composition may be prepared from the raw materials comprising any one of (b11) to (b15):

(b11) 60-80 parts by weight of rhizoma curcumae longae, 50-80 parts by weight of fructus Piperis longi, 30-60 parts by weight of ginseng, 30-60 parts by weight of mulberry, 25-40 parts by weight of Radix puerariae, 18-40 parts by weight of Cornus officinalis, 18-40 parts by weight of Semen ziziphi spinosae, 30-40 parts by weight of Radix astragali, 20-40 parts by weight of peach kernel, 15-20 parts by weight of licorice, and 15-20 parts by weight of honeysuckle;

(b12) 60-80 parts by weight of rhizoma curcumae longae, 50-60 parts by weight of Dendrobium nobile, 30-60 parts by weight of ginseng, 30-60 parts by weight of mulberry, 25-40 parts by weight of Radix puerariae, 18-40 parts by weight of Cornus officinalis, 18-40 parts by weight of Semen ziziphi spinosae, 30-40 parts by weight of Radix astragali, 20-40 parts by weight of peach kernel, 15-20 parts by weight of licorice, and 15-20 parts by weight of honeysuckle;

(b13) 60-80 parts by weight of rhizoma curcumae longae, 50-60 parts by weight of Dendrobium nobile, 50-80 parts by weight of fructus Piperis longi, 30-60 parts by weight of ginseng, 30-60 parts by weight of mulberry, 25-40 parts by weight of Radix puerariae, 18-40 parts by weight of Cornus officinalis, 18-40 parts by weight of Semen ziziphi spinosae, 30-40 parts by weight of Radix astragali, 20-40 parts by weight of peach kernel, 15-20 parts by weight of licorice, and 15-20 parts by weight of honeysuckle;

(b14) 50-60 parts by weight of Dendrobium nobile, 50-80 parts by weight of fructus Piperis longi, 30-60 parts by weight of ginseng, 30-60 parts by weight of mulberry, 25-40 parts by weight of Radix puerariae, 18-40 parts by weight of Cornus officinalis, 18-40 parts by weight of Semen ziziphi spinosae, 30-40 parts by weight of Radix astragali, 20-40 parts by weight of peach kernel, 15-20 parts by weight of licorice, and 15-20 parts by weight of honeysuckle; and

(b15) 60-80 parts by weight of rhizoma curcumae longae, 30-60 parts by weight of ginseng, 30-60 parts by weight of mulberry, 25-40 parts by weight of Radix puerariae, 18-40 parts by weight of Cornus officinalis, 18-40 parts by weight of Semen ziziphi spinosae, 30-40 parts by weight of Radix astragali, 20-40 parts by weight of peach kernel, 15-20 parts by weight of licorice, and 15-20 parts by weight of honeysuckle.

Preferably, the Dendrobium nobile may be Dendrobium officinale.

The third aspect of the invention provides a preparation method of the Chinese herbal medicine composition in the second aspect.

When the Chinese herbal medicine composition is prepared from any one of the raw materials from (c7) to (c10), a preparation method of the Chinese herbal medicine composition includes: conducting water extraction and/or alcohol extraction of the raw materials to obtain the Chinese herbal medicine composition;

(c7) rhizoma curcumae longae and fructus Piperis longi;

(c8) rhizoma curcumae longae and Dendrobium nobile;

(c9) rhizoma curcumae longae, Dendrobium nobile and fructus Piperis longi; and

(c10) Dendrobium nobile and fructus Piperis longi.

Preferably, the preparation method may include the following steps: 1) mixing raw materials with water and conducting an extraction and a solid-liquid separation to obtain a filtrate E and a medicine residue; 2) mixing the medicine residue obtained in step 1) with ethanol, and conducting an extraction and a solid-liquid separation to obtain a filtrate F; and 3) mixing the filtrate E and the filtrate F to obtain the Chinese herbal medicine composition.

Preferably, the raw materials in step 1) may be crushed and sieved through a 60-mesh sieve before mixing.

Preferably, a mass ratio of the raw materials to water in step 1) may be 1:(10 to 20).

Preferably, the extraction in step 1) may be a subcritical extraction, a decoction extraction, an ultrasonic extraction, a reflux extraction or a percolation extraction; preferably a subcritical extraction.

Preferably, the raw materials in step 1) may be steamed with a water vapor before mixing.

Preferably, the extraction in step 1) may be conducted at 160 to 200° C. and 2 to 4 MPa for 30 to 90 min; preferably at 160 to 180° C. and 3 to 4 MPa for 30 to 60 min.

Preferably, the extraction in step 1) may be conducted for 1 to 3 times.

Preferably, the mass-volume ratio (g/mL) of the medicine residue to the ethanol in step 2) may be 1:(15 to 35); preferably 1:(15 to 25).

Preferably, the ethanol may have a concentration of 40 to 70 v/v %; preferably 40 to 60 v/v %.

Preferably, the extraction in step 2) may be in a subcritical extraction, a decoction extraction, an ultrasonic extraction, a reflux extraction or a percolation extraction; preferably a decoction extraction.

Preferably, the extraction in step 2) may be conducted at 60 to 90° C. for 1 to 3 h.

Preferably, the extraction in step 2) may be conducted for 1 to 3 times.

Preferably, the preparation method may further include the following steps: conducting primary concentration, homogenization, secondary concentration and drying of a mixture of the filtrate E and the filtrate F.

Preferably, the primary concentration may be conducted at 60 to 75° C. and −0.3 to −0.5 MPa (a vacuum degree), and the resulted volume may be ⅓ to ⅙ of the volume of the mixture after concentration.

Preferably, the homogenization may be conducted at 80 to 120 MPa and 30 to 40° C. for 0.3 to 1 h.

Preferably, the homogenization may be conducted for 4 to 6 times.

Preferably, the secondary concentration may be conducted 60 to 75° C. and −0.3 to −0.5 MPa (a vacuum degree), and the resulted density may be a relative density of 1.2 to 1.5 after concentration.

Preferably, the drying may include at least one of spray-drying, freeze-drying, vacuum-drying and air-blast drying.

When the Chinese herbal medicine composition is prepared from any one of the raw materials from (c11) to (c15), a preparation method of the Chinese herbal medicine composition includes: conducting water extraction and/or alcohol extraction of the raw materials to obtain the Chinese herbal medicine composition;

(c11) rhizoma curcumae longae, fructus Piperis longi, ginseng, mulberry, Radix puerariae, Cornus officinalis, Semen ziziphi spinosae, Radix astragali, peach kernel, licorice, and honeysuckle;

(c12) rhizoma curcumae longae, Dendrobium nobile, ginseng, mulberry, Radix puerariae, Cornus officinalis, Semen ziziphi spinosae, Radix astragali, peach kernel, licorice, and honeysuckle;

(c13) rhizoma curcumae longae, Dendrobium nobile, fructus Piperis longi, ginseng, mulberry, Radix puerariae, Cornus officinalis, Semen ziziphi spinosae, Radix astragali, peach kernel, licorice, and honeysuckle;

(c14) Dendrobium nobile, fructus Piperis longi, ginseng, mulberry, Radix puerariae, Cornus officinalis, Semen ziziphi spinosae, Radix astragali, peach kernel, licorice, and honeysuckle; and

(c15) rhizoma curcumae longae, ginseng, mulberry, Radix puerariae, Cornus officinalis, Semen ziziphi spinosae, Radix astragali, peach kernel, licorice, and honeysuckle.

Preferably, the preparation method may include the following steps: 1) mixing raw materials C with water and conducting an extraction and a solid-liquid separation to obtain a filtrate C and a medicine residue; 2) mixing the medicine residue obtained in step 1) and raw materials D with ethanol, and conducting an extraction and a solid-liquid separation to obtain a filtrate D; and 3) mixing the filtrate C and the filtrate D to obtain a Chinese herbal medicine composition;

The raw materials C can be any one of (d11) to (d15):

(d11) rhizoma curcumae longae, fructus Piperis longi, mulberry, licorice, peach kernel, Semen ziziphi spinosae, and honeysuckle;

(d12) rhizoma curcumae longae, mulberry, licorice, peach kernel, Dendrobium nobile, Semen ziziphi spinosae, and honeysuckle;

(d13) rhizoma curcumae longae, fructus Piperis longi, ginseng, mulberry, licorice, peach kernel, Dendrobium nobile, Semen ziziphi spinosae, and honeysuckle;

(d14)fructus Piperis longi, ginseng, mulberry, licorice, peach kernel, Dendrobium nobile, Semen ziziphi spinosae, and honeysuckle; and

(d15) rhizoma curcumae longae, mulberry, licorice, peach kernel, Semen ziziphi spinosae, and honeysuckle; and

The raw materials D are Radix puerariae, ginseng, Radix astragali and Cornus officinalis.

Preferably, the raw materials C in step 1) may be crushed and sieved through a 60-mesh sieve before mixing.

Preferably, a mass ratio of the raw materials C to water in step 1) may be 1:(10 to 20).

Preferably, the extraction in step 1) may be a subcritical extraction, a decoction extraction, an ultrasonic extraction, a reflux extraction or a percolation extraction; preferably a subcritical extraction.

Preferably, the raw materials C in step 1) may be steamed with a water vapor before mixing.

Preferably, the extraction in step 1) may be conducted at 160 to 200° C. and 2 to 4 MPa for 30 to 90 min; preferably at 160 to 180° C. and 3 to 4 MPa for 30 to 60 min.

Preferably, the extraction in step 1) may be conducted for 1 to 3 times.

Preferably, the mass-volume ratio (g/mL) of the medicine residue to the ethanol in step 2) may be 1:(15 to 35); preferably 1:(15 to 25).

Preferably, the ethanol may have a concentration of 40 to 70 v/v %; preferably 40 to 60 v/v %.

Preferably, the extraction in step 2) may be a subcritical extraction, a decoction extraction, an ultrasonic extraction, a reflux extraction or a percolation extraction; preferably, a decoction extraction.

Preferably, the extraction in step 2) may be conducted at 60 to 90° C. for 1 to 3h.

Preferably, the extraction in step 2) may be conducted for 1 to 3 times.

Preferably, the preparation method may further include the following steps: conducting primary concentration, homogenization, secondary concentration and drying of a mixture of the filtrate C and the filtrate D.

Preferably, the primary concentration may be conducted at 60 to 75° C. and −0.3 to −0.5 MPa (a vacuum degree), and the resulted volume may be ⅓ to ⅙ of the volume of the mixture after concentration.

Preferably, the homogenization may be conducted at 80 to 120 MPa and 30 to 40° C. for 0.3 to 1 h.

Preferably, the homogenization may be conducted for 4 to 6 times.

Preferably, the secondary concentration may be conducted 60 to 75° C. and −0.3 to −0.5 MPa (a vacuum degree), and the resulted density may be a relative density of 1.2 to 1.5 after concentration.

Preferably, the drying may include at least one of spray-drying, freeze-drying, vacuum-drying and air-blast drying.

The fourth aspect of the invention provides use of the Chinese herbal medicine composition in the second aspect in preparing a product. Preferably, the product may include foods and a medicine.

The product has any one of functions from (1) to (6):

(1) preventing and/or treating hearing loss;

(2) reducing an ABR value;

(3) reducing the content of malondialdehyde;

(4) reducing the content of glutamic acid;

(5) reducing the content of IL-4; and

(6) inhibiting production of inflammatory factors.

Preferably, inflammatory factors may include TNF-α and NO; and preferably NO.

Preferably, the food may be a functional food.

Preferably, the type of the food may include a beverage, a biscuit, a candy and a pastry.

Preferably, the beverage may be a solid beverage.

Preferably, a dosage form of the medicine includes a capsule, a tablet, a granule, a mixture, a decoction, a pill, an electuary, a dripping pill, a liniment, a tincture and a wine.

Preferably, the product may also include a pharmaceutically acceptable auxiliary material or a food-acceptable auxiliary material.

Preferably, the hearing loss may include aging hearing loss, noise-induced hearing loss, and inflammatory hearing loss.

The fifth aspect of the present invention provides a product and the product includes the Chinese herbal medicine composition in the second aspect of the present invention.

Preferably, the type of the product may include a food and a medicine.

The product has any one of functions from (1) to (6):

(1) preventing and/or treating hearing loss;

(2) reducing an ABR value;

(3) reducing the content of malondialdehyde;

(4) reducing the content of glutamic acid;

(5) reducing the content of IL-4; and

(6) inhibiting production of inflammatory factors.

Preferably, inflammatory factors may include TNF-α and NO; preferably NO.

Preferably, the food may be a functional food.

Preferably, the type of the food may include a beverage, a biscuit, a candy and a pastry.

Preferably, the beverage may be a solid beverage.

Preferably, a dosage form of the medicine includes a capsule, a tablet, a granule, a mixture, a decoction, a pill, an electuary, a dripping pill, a liniment, a tincture and a wine.

Preferably, the product may also include a pharmaceutically acceptable auxiliary material or a food-acceptable auxiliary material.

Preferably, the hearing loss may include aging hearing loss, noise-induced hearing loss, and inflammatory hearing loss.

The sixth aspect of the present invention provides use of the Chinese herbal medicine composition in the second aspect of the present invention and/or the product in the fifth aspect in treating hearing loss.

Preferably, the use may include the following steps: administering an effective amount of the Chinese herbal medicine composition in the second aspect and/or the product in the fifth aspect to a patient.

Preferably, the Chinese herbal medicine composition may have a dosage of 0.5 to 4.0 g/d.

Preferably, the product may have a dosage of 0.5 to 4.0 g/d according to the Chinese herbal medicine composition.

Preferably, the Chinese herbal medicine composition may be administered orally, sublingually, intravenously, intramuscularly, subcutaneously, in an inhaling manner, and transdermally.

Preferably, the product may be administered orally, sublingually, intravenously, intramuscularly, subcutaneously, in an inhaling manner, and transdermally.

Preferably, the hearing loss may include aging hearing loss, noise-induced hearing loss, and inflammatory hearing loss.

Beneficial effects of the present invention are as follows:

The invention discloses for the first time use of rhizoma curcumae longae in preventing and/or treating hearing loss. The rhizoma curcumae longae can reduce the ABR value, the content of malondialdehyde, the content of glutamic acid and the content of IL-4, and inhibit production of inflammatory factors to achieve an effect of preventing and/or treating hearing loss.

The invention provides a Chinese herbal medicine composition. The Chinese herbal medicine composition is prepared from at least two of medicinal and edible raw materials of rhizoma curcumae longae, Dendrobium nobile and fructus Piperis longi, can effectively reduce the content of NO secreted by cells, reduces cell inflammation, and has a good effect of relieving hearing loss induced by lipopolysaccharide (LPS). The Chinese herbal medicine composition is not a simple combination of traditional Chinese medicines and has a synergistic effect. At the same time, the Chinese herbal medicine composition can significantly reduce the ABR value (p<0.05), the malondialdehyde (MDA) (p<0.05), and the glutamic acid (p<0.05) of a patient with hearing loss (aging hearing loss, noise-induced hearing loss, and inflammatory hearing loss), significantly relieves hearing loss, and can be developed into functional foods and medicines for relieving hearing loss as an active ingredient.

Furthermore, the medicinal and edible raw materials of ginseng, mulberry, fructus Piperis longi, ginseng, Radix puerariae, Cornus officinalis, Semen ziziphi spinosae, Radix astragali, peach kernel, licorice, and honeysuckle are added, the obtained Chinese herbal medicine composition can extremely significantly reduce the ABR value (p<0.001), the MDA (p<0.001), the glutamic acid (p<0.001), and IL-4 (p<0.001) of a patient with hearing loss (aging hearing loss, noise-induced hearing loss, and inflammatory hearing loss) and extremely significantly relieves hearing loss. The Chinese herbal medicine composition has a better effect than antideaf otic pills. Besides, aiming at different hearing loss, the provided Chinese herbal medicine composition (rhizoma curcumae longae, Dendrobium nobile, fructus Piperis longi, ginseng, mulberry, Radix puerariae, Cornus officinalis, Semen ziziphi spinosae, Radix astragali, peach kernel, licorice, and honeysuckle) has more effective effects on reducing the ABR and/or the MDA and/or the glutamic acid and/or the IL-4 than the sum of the effect of each component. The Chinese herbal medicine composition has a synergistic effect and can be developed into functional foods and medicines for relieving hearing loss as an active ingredient.

Aiming at hearing loss of people caused by environmental noise, body aging and inflammation in daily life, the medicinal and edible raw materials of the rhizoma curcumae longae, Dendrobium nobile, ginseng, mulberry, fructus Piperis longi, Radix puerariae, Cornus officinalis, Semen ziziphi spinosae, Radix astragali, peach kernel, licorice, and honeysuckle are combined to be developed into medicinal and edible functional foods and medicine. The developed product can be used for relieving subhealth of ears, such as aging hearing loss and hearing loss of people working in noise environment, is suitable for a variety of induced hearing loss, and can reduce side effects caused by long-term use of medicines in people with hearing loss. In addition, the combined raw materials of the rhizoma curcumae longae, Dendrobium nobile and fructus Piperis longi significantly increase a hearing loss-relieving function of the combination of the ginseng, mulberry, Radix puerariae, Cornus officinalis, Semen ziziphi spinosae, Radix astragali, peach kernel, licorice, and honeysuckle, and the increase of the components has a synergistic effect, not a simple superimposed effect. At the same time, the raw materials, content and technology used in the present invention can relieve bitter and pungent tastes of the prescription extract, reduces unnecessary sweeteners and correctives in the finished product, and helps to improve an eating effect of consumers.

### DETAILED DESCRIPTION

The invention will be further described in detail below with specific examples.

It should be understood that these examples are only used to illustrate the present invention and not to limit the scope of the present invention.

The experimental methods not indicated with specific conditions in the following examples are usually conducted conventionally or according to recommendation by a manufacturer. Unless otherwise specified, the materials, reagents, etc. used in the examples are commercially available.

### Example 1: Chinese Herbal Medicine Extract for Relieving Hearing Loss

1. A Chinese herbal medicine extract for relieving hearing loss was prepared from the following raw materials in parts by weight: 80 parts of rhizoma curcumae longae.

2. A preparation method of the Chinese herbal medicine extract for relieving hearing loss included the following steps:

S1. a dried rhizoma curcumae longae product was crushed and sieved through a 60-mesh sieve, the medicinal fine powder was measured according to the formula amount and put into a subcritical extraction tank for steaming for 5 min with a water vapor, water at 15 times the mass of the medicinal material was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for late use;

S2. the medicine residue of step 51 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at a rotating drum speed of 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine extract with the moisture content of 3.28%.

### Example 2: Chinese Herbal Medicine Composition for Relieving Hearing Loss

1. A Chinese herbal medicine composition for relieving hearing loss was prepared from the following raw materials in parts by weight: 80 parts of rhizoma curcumae longae and 80 parts of fructus Piperis longi.

2. A preparation method of the Chinese herbal medicine composition for relieving hearing loss included the following steps:

S1. a dried rhizoma curcumae longae product and a dried fructus Piperis longi product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for late use;

S2. the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine composition with the moisture content of 2.99 w/w %.

### Example 3: Chinese Herbal Medicine Composition for Relieving Hearing Loss

1. A Chinese herbal medicine composition for relieving hearing loss was prepared from the following raw materials in parts by weight: 80 parts of rhizoma curcumae longae and 60 parts of Dendrobium officinale.

2. A preparation method of the Chinese herbal medicine composition for relieving hearing loss included the following steps:

S1. a dried rhizoma curcumae longae product and a dried Dendrobium officinale product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for late use;

S2. the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine composition with the moisture content of 3.27 w/w %.

### Example 4: Chinese Herbal Medicine Composition for Relieving Hearing Loss

1. A Chinese herbal medicine composition for relieving hearing loss was prepared from the following raw materials in parts by weight: 80 parts of fructus Piperis longi and 60 parts of Dendrobium officinale.

2. A preparation method of the Chinese herbal medicine composition for relieving hearing loss included the following steps:

S1. a dried fructus Piperis longi product and a dried Dendrobium officinale product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for late use;

S2. the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine composition with the moisture content of 3.54 w/w %.

### Example 5: Chinese Herbal Medicine Composition for Relieving Hearing Loss

1. A Chinese herbal medicine composition for relieving hearing loss was prepared from the following raw materials in parts by weight: 80 parts of rhizoma curcumae longae, 80 parts of fructus Piperis longi and 60 parts of Dendrobium officinale.

2. A preparation method of the Chinese herbal medicine composition for relieving hearing loss included the following steps:

S1. a dried rhizoma curcumae longae product, a dried fructus Piperis longi product and a dried Dendrobium officinale product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 MPa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine composition with the moisture content of 4.20 w/w %.

### Example 6: Chinese Herbal Medicine Composition for Relieving Hearing Loss

1. A Chinese herbal medicine composition for relieving hearing loss was prepared from the following raw materials in parts by weight: 80 parts of rhizoma curcumae longae, 80 parts of fructus Piperis longi, 60 parts of Dendrobium officinale, 60 parts of ginseng, 60 parts of mulberry, 40 parts of Radix puerariae, 40 parts of Cornus officinalis, 40 parts of Semen ziziphi spinosae, 40 parts of Radix astragali, 40 parts of peach kernel, 20 parts of licorice, and 20 parts of honeysuckle.

2. A preparation method of the Chinese herbal medicine composition for relieving hearing loss included the following steps:

S1. a dried rhizoma curcumae longae product, a dried fructus Piperis longi product, a dried mulberry product, a dried licorice product, a dried peach kernel product, a dried Dendrobium officinale product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.35 MPa to a relative density of 1.30, and drying was conducted to obtain the Chinese herbal medicine composition with the moisture content of 3.47 w/w %.

### Example 7: Chinese Herbal Medicine Composition for Relieving Hearing Loss

1. A Chinese herbal medicine composition for relieving hearing loss was prepared from the following raw materials in parts by weight: 40 parts of rhizoma curcumae longae, 40 parts of fructus Piperis longi, 30 parts of Dendrobium officinale, 20 parts of ginseng, 20 parts of mulberry, 10 parts of Radix puerariae, 10 parts of Cornus officinalis, 10 parts of Semen ziziphi spinosae, 10 parts of Radix astragali, 10 parts of peach kernel, 5 parts of licorice, and 5 parts of honeysuckle.

2. A preparation method of the Chinese herbal medicine composition for relieving hearing loss included the following steps:

S1. a dried rhizoma curcumae longae product, a dried fructus Piperis longi product, a dried mulberry product, a dried licorice product, a dried peach kernel product, a dried Dendrobium officinale product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 160° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step S1 was put into the extraction tank, an aqueous solution with ethanol at a concentration of 40% (a material-to-liquid ratio (g/mL) of the medicine residue to the solution was 1:15) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.3 MPa to a relative density of 1.30, and drying was conducted to obtain the Chinese herbal medicine composition with the moisture content of 4.04 w/w %.

### Example 8: Chinese Herbal Medicine Composition for Relieving Hearing Loss

1. A Chinese herbal medicine composition for relieving hearing loss was prepared from the following raw materials in parts by weight: 50 parts of rhizoma curcumae longae, 50 parts of fructus Piperis longi, 40 parts of Dendrobium officinale, 40 parts of ginseng, 40 parts of mulberry, 25 parts of Radix puerariae, 20 parts of Cornus officinalis, 20 parts of Semen ziziphi spinosae, 20 parts of Radix astragali, 20 parts of peach kernel, 8 parts of licorice, and 8 parts of honeysuckle.

2. A preparation method of the Chinese herbal medicine composition for relieving hearing loss included the following steps:

S1. a dried rhizoma curcumae longae product, a dried fructus Piperis longi product, a dried mulberry product, a dried licorice product, a dried peach kernel product, a dried Dendrobium officinale product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 170° C. and 3 MPa for 45 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step S1 was put into the extraction tank, an aqueous solution with ethanol at a concentration of 50% (a material-to-liquid ratio (g/mL) of the medicine residue to the solution was 1:20) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.3 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.4 MPa to a relative density of 1.30, and drying was conducted to obtain the Chinese herbal medicine composition with the moisture content of 2.98 w/w %.

### Example 9: Chinese Herbal Medicine Composition for Relieving Hearing Loss

1. A Chinese herbal medicine composition for relieving hearing loss was prepared from the following raw materials in parts by weight: 60 parts of rhizoma curcumae longae, 50 parts of fructus Piperis longi, 50 parts of Dendrobium officinale, 30 parts of ginseng, 30 parts of mulberry, 40 parts of Radix puerariae, 18 parts of Cornus officinalis, 18 parts of Semen ziziphi spinosae, 30 parts of Radix astragali, 20 parts of peach kernel, 15 parts of licorice, and 15 parts of honeysuckle.

2. A preparation method of the Chinese herbal medicine composition for relieving hearing loss included the following steps:

S1. a dried rhizoma curcumae longae product, a dried fructus Piperis longi product, a dried mulberry product, a dried licorice product, a dried peach kernel product, a dried Dendrobium officinale product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 165° C. and 3 MPa for 50 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step 51 was put into the extraction tank, an aqueous solution with ethanol at a concentration of 48% (a material-to-liquid ratio (g/mL) of the medicine residue to the solution was 1:20) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.4 MPa to a relative density of 1.30, and drying was conducted to obtain the Chinese herbal medicine composition with the moisture content of 3.21 w/w %.

### Example 10: Chinese Herbal Medicine Composition for Relieving Hearing Loss

1. A Chinese herbal medicine composition for relieving hearing loss was prepared from the following raw materials in parts by weight: 80 parts of rhizoma curcumae longae, 60 parts of ginseng, 60 parts of mulberry, 40 parts of Radix puerariae, 40 parts of Cornus officinalis, 40 parts of Semen ziziphi spinosae, 40 parts of Radix astragali, 40 parts of peach kernel, 20 parts of licorice, and 20 parts of honeysuckle.

2. A preparation method of the Chinese herbal medicine composition for relieving hearing loss included the following steps:

S1. a dried rhizoma curcumae longae product, a dried mulberry product, a dried licorice product, a dried peach kernel product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.3 MPa to a relative density of 1.30, and drying was conducted to obtain the Chinese herbal medicine composition with the moisture content of 2.76 w/w %.

### Example 11: Chinese Herbal Medicine Composition for Relieving Hearing Loss

1. A Chinese herbal medicine composition for relieving hearing loss was prepared from the following raw materials in parts by weight: 80 parts of rhizoma curcumae longae, 80 parts of fructus Piperis longi, 60 parts of ginseng, 60 parts of mulberry, 40 parts of Radix puerariae, 40 parts of Cornus officinalis, 40 parts of Semen ziziphi spinosae, 40 parts of Radix astragali, 40 parts of peach kernel, 20 parts of licorice, and 20 parts of honeysuckle.

2. A preparation method of the Chinese herbal medicine composition for relieving hearing loss included the following steps:

S1. a dried rhizoma curcumae longae product, a dried fructus Piperis longi product, a dried mulberry product, a dried licorice product, a dried peach kernel product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step 51 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.4 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.4 MPa to a relative density of 1.30, and drying was conducted to obtain the Chinese herbal medicine composition with the moisture content of 2.39 w/w %.

### Example 12: Chinese Herbal Medicine Composition for Relieving Hearing Loss

1. A Chinese herbal medicine composition for relieving hearing loss was prepared from the following raw materials in parts by weight: 80 parts of rhizoma curcumae longae, 60 parts of Dendrobium officinale, 60 parts of ginseng, 60 parts of mulberry, 40 parts of Radix puerariae, 40 parts of Cornus officinalis, 40 parts of Semen ziziphi spinosae, 40 parts of Radix astragali, 40 parts of peach kernel, 20 parts of licorice, and 20 parts of honeysuckle.

2. A preparation method of the Chinese herbal medicine composition for relieving hearing loss included the following steps:

S1. a dried rhizoma curcumae longae product, a dried Dendrobium officinale product, a dried mulberry product, a dried licorice product, a dried peach kernel product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.4 Mpa to ⅕ of the volume of the mixture, and an obtained concentrated solution was subjected to a high-pressure homogenization at 100 Mpa and 35° C. for 0.5 h for a total of 5 times to obtain a traditional Chinese medicine mixture; and

S4. the traditional Chinese medicine mixture of step S3 was concentrated at 65° C. and −0.4 MPa to a relative density of 1.30, and drying was conducted to obtain the Chinese herbal medicine composition with the moisture content of 3.41 w/w %.

### Example 13: Chinese Herbal Medicine Composition for Relieving Hearing Loss

1. A Chinese herbal medicine composition for relieving hearing loss was prepared from the following raw materials in parts by weight: 60 parts of Dendrobium officinale, 80 parts of fructus Piperis longi, 60 parts of ginseng, 60 parts of mulberry, 40 parts of Radix puerariae, 40 parts of Cornus officinalis, 40 parts of Semen ziziphi spinosae, 40 parts of Radix astragali, 40 parts of peach kernel, 20 parts of licorice, and 20 parts of honeysuckle.

2. A preparation method of the Chinese herbal medicine composition for relieving hearing loss included the following steps:

S1. a dried Dendrobium officinale product, a dried fructus Piperis longi product, a dried mulberry product, a dried licorice product, a dried peach kernel product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step 51 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine composition with the moisture content of 3.35 w/w %.

### Comparative Example 1: Chinese Herbal Medicine Extract for Relieving Hearing Loss

1. A Chinese herbal medicine composition for relieving hearing loss was prepared from the following raw materials in parts by weight: 80 parts of fructus Piperis longi.

2. A preparation method of the Chinese herbal medicine composition for relieving hearing loss included the following steps:

S1. a dried fructus Piperis longi product was crushed and sieved through a 60-mesh sieve, the medicinal fine powder was measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine composition with the moisture content of 2.62 w/w %.

### Comparative Example 2: Chinese Herbal Medicine Extract for Relieving Hearing Loss

1. A Chinese herbal medicine composition for relieving hearing loss was prepared from the following raw materials in parts by weight: 60 parts of Dendrobium officinale.

2. A preparation method of the Chinese herbal medicine composition for relieving hearing loss included the following steps:

S1. a dried Dendrobium officinale product was crushed and sieved through a 60-mesh sieve, the medicinal fine powder was measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for late use;

S2. the medicine residue of step S1 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.5 MPa to a relative density of 1.30, and drying was conducted to obtain the Chinese herbal medicine composition with the moisture content of 4.11 w/w %.

### Comparative Example 3: Chinese Herbal Medicine Composition for Relieving Hearing Loss

1. A Chinese herbal medicine composition for relieving hearing loss was prepared from the following raw materials in parts by weight: 60 parts of ginseng, 60 parts of mulberry, 40 parts of Radix puerariae, 40 parts of Cornus officinalis, 40 parts of Semen ziziphi spinosae, 40 parts of Radix astragali, 40 parts of peach kernel, 20 parts of licorice, and 20 parts of honeysuckle.

2. A preparation method of the Chinese herbal medicine composition for relieving hearing loss included the following steps:

S1. a dried mulberry product, a dried licorice product, a dried peach kernel product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step 51 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.3 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.4 MPa to a relative density of 1.30, and drying was conducted to obtain the Chinese herbal medicine composition with the moisture content of 3.06 w/w %.

### Comparative Example 4: Chinese Herbal Medicine Composition for Relieving Hearing Loss

1. A Chinese herbal medicine composition for relieving hearing loss was prepared from the following raw materials in parts by weight: 80 parts of fructus Piperis longi, 60 parts of ginseng, 60 parts of mulberry, 40 parts of Radix puerariae, 40 parts of Cornus officinalis, 40 parts of Semen ziziphi spi by weight of licorice, and 20 parts of honeysuckle.

2. A preparation method of the Chinese herbal medicine composition for relieving hearing loss included the following steps:

S1. a dried fructus Piperis longi product, a dried mulberry product, a dried licorice product, a dried peach kernel product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step 51 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.4 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.4 MPa to a relative density of 1.30, and drying was conducted to obtain the Chinese herbal medicine composition with the moisture content of 3.76 w/w %.

### Comparative Example 5: Chinese Herbal Medicine Composition for Relieving Hearing Loss

1. A Chinese herbal medicine composition for relieving hearing loss was prepared from the following raw materials in parts by weight: 60 parts of Dendrobium officinale, 60 parts of ginseng, 60 parts of mulberry, 40 parts of Radix puerariae, 40 parts of Cornus officinalis, 40 parts of Semen ziziphi spinosae, 40 parts of Radix astragali, 40 parts of peach kernel, 20 parts of licorice, and 20 parts of honeysuckle.

2. A preparation method of the Chinese herbal medicine composition for relieving hearing loss included the following steps:

S1. a dried Dendrobium officinale product, a dried mulberry product, a dried licorice product, a dried peach kernel product, a dried Semen ziziphi spinosae product and a dried honeysuckle product were separately crushed and sieved through a 60-mesh sieve, the medicinal fine powders were measured according to the formula amount and put into a subcritical extraction tank for steaming for 5 min with a water vapor, water at 15 times the mass of the medicinal materials was added, an extraction was conducted at 180° C. and 3 MPa for 60 min, after a temperature was lowered to a room temperature, filtration and centrifugation were conducted to obtain a water extract, the extraction was repeated once to obtain a filtrate, and a medicine residue was for later use;

S2. Radix puerariae, ginseng, Radix astragali and Cornus officinalis were crushed and sieved through a 60-mesh sieve, the medicinal materials were put into the extraction tank according to the formula amount, the medicine residue of step 51 was put into the extraction tank, an aqueous solution with ethanol at a concentration of 60% (a material-to-liquid ratio (g/mL) of the medicine residue to the solution was 1:25) was added, an extraction was conducted at 70° C. for 2 h, filtration was conducted, the extraction was repeated once, a filter residue was discarded, an extract was filtered with a tubular centrifuge (at 20,000 r/min) and a filtrate was collected for later use;

S3. the filtrates of steps S1 and S2 were mixed, an obtained mixture was concentrated at 65° C. and −0.5 Mpa to ⅕ of the volume of the mixture, and an obtained concentrated solution was subjected to a high-pressure homogenization at 100 MPa and 35° C. for 0.5 h for a total of 5 times to obtain a traditional Chinese medicine mixture; and

S4. the traditional Chinese medicine mixture of step S3 was concentrated at 65° C. and −0.4 MPa to a relative density of 1.30, and drying was conducted to obtain the Chinese herbal medicine composition with the moisture content of 4.45 w/w %.

### Effect Examples

### 1. Effect of Chinese Herbal Medicine Extract/Chinese Herbal Medicine Composition on Lipopolysaccharide (LPS)-Induced Nitric Oxide Secretion in RAW264.7 Cells

After lipopolysaccharide (LPS) inducer acts on the cochlea, the LPS inducer stimulates and causes a series of inflammatory reactions in a cochlear tissue. Specifically, the LPS inducer induces cochlear hearing loss, at the same time promotes over expressions of inflammatory factors such as TNF-α and nitric oxide (NO), and induces an oxidation stress response of the cochlear tissue, and exacerbates hearing loss. Therefore, in a modeling, abnormal secretion of NO in inflammation can also reflect the degree of the hearing loss. RAW264.7 cells (RAW264.7 mouse macrophages) were inoculated on a 6-well plate at a density of 2×10⁶ cells/mL and cultured for 24 h until the cells adhered; a complete medium was used in a blank group, LPS with a final mass concentration of 2 mg/L was used in a model group, and a test sample (the Chinese herbal medicine extract/Chinese herbal medicine composition of examples 1 to 5 and comparative examples 1 to 2) with a final mass concentration of 100 μg/mL and 2 mg/L of LPS were separately used in each administration group (sample group). The total volume of each group was 2 mL and each group was put in a cell incubator to be cultured continuously for 24 h; and a cell supernatant was collected and measured according to the instructions of a NO detection kit, each experiment was independently repeated 3 times, and the inhibition ratio (IR) of the NO was calculated, where S was the NO content of each group.

IR (%)=(S_{model group}−S_{sample group})/(S_{model group}−S_{blank group})×100%

A synergic index of each component was calculated according to the content of the index value (NO) and the synergic index (Berenbaum index) was calculated as follows: a calculation formula of the reference (Berenbaum M C. The expected effect of a combination of agents: the general solution. Journal of Theoretical Biology, 1985, 114:413-431):

$${\sum }_{i = 1}^{n} \frac{X i}{X i e} ,$$

where Xi referred to dosage of an i-th medicine when used in combination; Xie referred to dosage of the i-th medicine that can produce the same effect as the combination when used alone; n referred to the number of medicines used in combination; and when the Berenbaum index was less than 1, the medicine combination was synergistic and when the Berenbaum index was greater than 1, the medicine combination was antagonistic.

The experimental results were shown in Table 1: LPS induced abnormal NO secretion after acting on the RAW264.7 cells, and the NO content of the cells in the model group increased significantly, indicating that the model was successful; after the Chinese herbal medicine extract/Chinese herbal medicine composition prepared from at least one of rhizoma curcumae longae, Dendrobium officinale and fructus Piperis longi acted on the LPS-induced cells, the NO content of the cells was effectively reduced and cell inflammation was reduced, indicating that the Chinese herbal medicine extract/Chinese herbal medicine composition prepared from at least one of rhizoma curcumae longae, Dendrobium officinale and fructus Piperis longi had a good effect of relieving LPS-induced hearing loss. The mechanism may lie in reducing the degree of the hearing loss by inhibiting the inflammatory process. However, there was no disclosure of use of rhizoma curcumae longae in relieving aging hearing loss in the prior art. In addition, the Chinese herbal composition provided in examples 2 to 5 had the synergic index of less than 1, indicating that the Chinese herbal composition provided in examples 2 to 5 was not a simple combination, but had a synergistic anti-inflammatory effect.

TABLE 1
Synergistic effect of each component

| Experiemntal | Content of | Inhibition | Synergic index |
|---|---|---|---|
| group | NO(μmol/L) | ratio of NO (%) | (SI*) |
| Blank group | 7.89 | | |
| Model group | 34.88 | | |
| Example 1 group | 24.39 | 38.87 | |
| Example 2 group | 19.42 | 57.29 | 0.4028 |
| Example 3 group | 23.46 | 42.31 | 0.6818 |
| Example 4 group | 24.88 | 37.04 | 0.8934 |
| Example 5 group | 12.86 | 81.58 | 0.3475 |
| Comparative | 27.50 | 27.33 | |
| example 1 group | | | |
| Comparative | 29.80 | 18.83 | |
| example 2 group | | | |

Note:
*referred to SI of the composition, a single raw material was used in example 1 and comparative examples 1-2 (the synergic index was not calculated), according to a determination index, SI value less than 1 indicated a synergistic effect produced by the composition, the smaller SI value meant that each component in the composition used at a lower dosage (concentration) can play an effect or exceed the effect produced by the same dosage of a single component (that is, the total concentration of the components of the composition), the better the synergistic effect presented, such that the combination of example 5 had the best synergistic effect, followed by a combination of rhizoma curcumae longae and fructus piperis longi (example 2), a combination of rhizoma curcumae longae and dendrobium officinale (example 3) and a combination of fructus piperis longi and dendrobium officinale (example 4).

### 2. Aging Hearing Loss

According to a method in the reference (Bai Xue, Liao Zhihang, Zhou Lijuan, et al. Effect of hearing-improving granules on hearing of experimental senile guinea pigs [J]. Chinese Traditional and Herbal Drugs, 2009, 40(5):777-779), a D-galactose-induced SD rat aging hearing loss model was used to evaluate the efficacy of the compositions of examples 1 to 5 and comparative examples 1 to 7. The specific method was as follows: the SD rats were adaptively reared for 7 days and divided into a blank group, a model group, a positive group (antideaf otic pills) and sample groups (example 5 to example 13, and comparative example 3 to comparative example 5), a total of 15 groups, each with 6 rats. Except for the blank group, the rats in the rest of the model group, the positive group and the sample groups were intraperitoneally injected with a D-galactose solution at a dosage of 500 mg/kg·d separately, and the modeling lasted for 8 weeks. From the first day of the D-galactose modeling, the rats in the positive group and the sample groups were gavaged with a dosage of 100 mg/kg of rats (0.3 mL/100 g rat body weight per day), and the rats in the blank group and the model group were given gavaged with the same amount of normal saline. The rats were weighed every week. In the last week of the experiment, the ABR value of each group of rats was detected by an auditory brainstem response (ABR) instrument under a condition of 8 kHZ. On the last day of the eighth week, after the ABR value of each group of rats was detected by the ABR instrument under a condition of 8 kHZ, the rats were sacrificed and cochlear tissues of the rats in each group were taken, and the contents of the MDA and the glutamic acid (GLU) in the cochlear tissues were detected. The results were shown in Table 2. Compared with the blank group, the ABR value, the MDA, and the glutamic acid of the rats in the model group were significantly different (all p<0.001), indicating that the model was successful; the ABR value (p<0.05), the MDA (p<0.05), and the glutamic acid (p<0.05) of the rats in the example 5, 10, and 13 groups were significantly different through the regulation, the effect was good, and there was no disclosure of use of rhizoma curcumae longae in relieving aging hearing loss in the prior art; the ABR value (p<0.01), the MDA (p<0.01), and the glutamic acid (p<0.01) of the rats in examples 11 and 12 groups were significantly different through the regulation; the ABR value (p<0.001), the MDA (p<0.001), and the glutamic acid (p<0.001) of the rats in example 6 to 9 groups were significantly different through regulation, and the effect was good and significantly better than those of the rats in comparative examples 3 to 5; the ABR value (p<0.001) and the glutamic acid (p<0.001) of the rats in the positive group were significantly different, but the MDA showed no significant difference, indicating that the compositions of examples 6 to 9 had a significant better effect than that of the positive group; especially, the reduction value of the GLU of the rats in the example 6 to 9 groups (the composition was prepared from rhizoma curcumae longae, fructus Piperis longi, Dendrobium officinale, ginseng, mulberry, Radix puerariae, Cornus officinalis, Semen ziziphi spinosae, Radix astragali, peach kernel, licorice, and honeysuckle) (21.95, 20.17, 23.53 and 21.72 mg/L, respectively) relative to the model group was all higher than the sum of that of the rats in example 5 (the composition was prepared from rhizoma curcumae longae, fructus Piperis longi and Dendrobium officinale) relative to the model group and that of the rats in comparative example 3 (the composition was prepared from ginseng, mulberry, Radix puerariae, Cornus officinalis, Semen ziziphi spinosae, Radix astragali, peach kernel, licorice and honeysuckle) to the model group (11.13+5.67=16.8 mg/L), thus the compositions of examples 6 to 9 had a synergistic effect in reducing the GLU; and the composition of example 9 had a synergistic effect in reducing the ABR and the compositions of examples 6 and 9 had a synergistic effect in reducing the MDA.

TABLE 2
Experimental results of aging hearing loss of rats

| Experimental group | ABRvalue (dB) | MDA(U/mL) | GLU(mg/L) |
|---|---|---|---|
| Blank group | 32.00 ± 3.38 | 5.30 ± 1.11 | 73.26 ± 1.39 |
| Model group | 44.71 ± 2.05^{###} | 14.18 ± 2.82^{###} | 93.80 ± 2.12^{###} |
| Positive group | 34.98 ± 1.23*** | 13.10 ± 1.58 | 73.00 ± 3.43*** |
| Example 5 group | 37.86 ± 3.18* | 10.16 ± 2.51* | 82.67 ± 2.13* |
| Example 6 group | 33.29 ± 3.45*** | 5.44 ± 1.61*** | 71.85 ± 2.77*** |
| Example 7 group | 33.43 ± 3.89*** | 7.32 ± 2.64*** | 73.63 ± 2.95*** |
| Example 8 group | 34.71 ± 4.03*** | 6.95 ± 2.91*** | 70.27 ± 3.93*** |
| Example 9 group | 32.03 ± 1.89*** | 6.27 ± 2.12*** | 72.08 ± 2.29*** |
| Example 10 group | 39.02 ± 1.10* | 10.03 ± 0.98* | 86.54 ± 1.38* |
| Example 11 group | 35.71 ± 1.31** | 9.10 ± 0.83** | 78.56 ± 1.39** |
| Example 12 group | 36.28 ± 2.27** | 8.27 ± 1.74** | 76.85 ± 1.33** |
| Example 13 group | 37.45 ± 2.03* | 8.68 ± 2.39* | 79.84 ± 2.41* |
| Comparative | 40.01 ± 2.25 | 10.74 ± 1.47* | 88.13 ± 1.79 |
| example 3 group | | | |
| Comparative | 38.56 ± 2.72* | 9.86 ± 1.90* | 83.07 ± 1.16* |
| example 4 group | | | |
| Comparative | 39.89 ± 3.38 | 11.11 ± 0.42 | 85.72 ± 0.44* |
| example 5 group | | | |

Note:
^{###}meant p < 0.001 compared to the blank group;
***meant p < 0.001 compared to the model group;
**meant p < 0.01 compared to the model group; and
*meant p < 0.05 compared to the model group.

### 3. Noise-Induced Hearing Loss

A noise modeling animal model was used for evaluation. The SD rats were adaptively reared for 7 days and divided into a blank group, a model group, a positive group (antideaf otic pills) and sample groups (example 5 to example 13, and comparative example 3 to comparative example 5), a total of 15 groups, each with 6 rats. Except for the blank group, the rats in the rest of the model group, the positive group and the sample groups were placed in cages with 6 SD rats in each cage and placed in an exposure chamber (with a soundproof box of 1.2 m long, 1.2 m wide and 1.7 m high). A white noise generator was used for sounding, an A-K200 power amplifier was used for amplification, and the noise was played to the exposure chamber by a speaker located on the top part of the exposure chamber. A sound pressure meter was used for continuous monitoring during exposure, the noise was maintained at a frequency of 20 Hz-20 kHz, an average sound intensity was a 90 dB sound pressure level, an unevenness of a sound field in an animal exposure range was (±2) dB, ventilation of the exposure chamber was ensured during the noise exposure, and the rats were given normal feed and normal drinking water and continuously exposed every day for 6 hours for a total of 14 d. During the modeling period, the rats in the positive group and the sample groups were gavaged with a dosage of 100 mg/kg of rats (0.3 mL/100 g rat body weight per day), and the rats in the blank group and the model group were given gavaged with the same amount of normal saline. At the end of the experiment (the 14th day), the ABR value of each group of rats was detected by the ABR instrument under a condition of 8 kHZ, after the detection, the cochlear tissues of the rats in each group were taken, and the contents of the MDA and the glutamic acid in the cochlear tissues were detected. The results were shown in Table 3. Compared with the blank group, the ABR value, the MDA, and the glutamic acid of the rats in the model group were significantly different (p<0.001/p<0.01), indicating that the model was successful; the ABR value (p<0.05), the MDA (p<0.05/p<0.01), and the glutamic acid (p<0.05/p<0.01) of the rats in the example 5 and 10 to 13 groups were significantly different through the regulation, the effect was good, and there was no disclosure of use of rhizoma curcumae longae in relieving noise-induced hearing loss in the prior art; the ABR value (p<0.001), the MDA (p<0.001), and the glutamic acid (p<0.001) of the rats in example 6 to 9 groups were significantly different through regulation, and the effect was good and significantly better than those of the rats in comparative examples 3 to 5; the ABR value (p<0.001), the glutamic acid (p<0.001) and the MDA (p<0.05) of the rats in the positive group were significantly different, but the compositions of examples 6 to 9 had a significant better effect than that of the positive group; especially, the reduction value of the MDA of the rats in the example 6 to 9 groups (the composition was prepared from rhizoma curcumae longae, fructus Piperis longi, Dendrobium officinale, ginseng, mulberry, Radix puerariae, Cornus officinalis, Semen ziziphi spinosae, Radix astragali, peach kernel, licorice, and honeysuckle) (17.63, 18.86, 17.53 and 20.7 U/mL respectively) relative to the model group was all higher than the sum of that of the rats in example 5 (the composition was prepared from rhizoma curcumae longae, fructus Piperis longi and Dendrobium officinale) relative to the model group and that of the rats in comparative example 3 (the composition was prepared from ginseng, mulberry, Radix puerariae, Cornus officinalis, Semen ziziphi spinosae, Radix astragali, peach kernel, licorice and honeysuckle) to the model group (8.95+6.18=15.13 U/mL), thus the compositions of examples 6 to 9 had a synergistic effect in reducing the MDA; and the compositions of examples, 6, 8 and 9 had a synergistic effect in reducing the ABR and the composition of example 9 had a synergistic effect in reducing the MDA.

TABLE 3
Experimental results of noise-induced hearing loss of rats

| Experimental group | ABR value (dB) | MDA(U/mL) | GLU(mg/L) |
|---|---|---|---|
| Blank group | 28.57 ± 2.26 | 62.06 ± 2.48 | 62.52 ± 1.24 |
| Model group | 41.43 ± 5.80^{##} | 73.08 ± 3.12^{##} | 79.84 ± 1.91^{##} |
| Positive group | 32.14 ± 2.47*** | 63.28 ± 1.86* | 57.29 ± 3.44*** |
| Example 5 group | 34.29 ± 1.23* | 64.13 ± 2.57* | 66.63 ± 3.13* |
| Example 6 group | 30.71 ± 3.19*** | 55.45 ± 1.81*** | 59.35 ± 1.35*** |
| Example 7 group | 32.86 ± 1.52*** | 54.22 ± 3.32*** | 55.34 ± 1.76*** |
| Example 8 group | 25.71 ± 1.75*** | 55.55 ± 1.43*** | 57.99 ± 3.60*** |
| Example 9 group | 28.17 ± 1.13*** | 52.38 ± 2.32*** | 54.12 ± 2.74*** |
| Example 10 group | 36.43 ± 1.25* | 64.04 ± 2.08* | 66.58 ± 1.81* |
| Example 11 group | 34.59 ± 0.31* | 61.45 ± 2.38** | 62.57 ± 1.88** |
| Example 12 group | 36.00 ± 1.11* | 62.61 ± 0.98** | 65.03 ± 4.08* |
| Example 13 group | 35.18 ± 0.27* | 63.20 ± 2.01* | 63.45 ± 2.20** |
| Comparative | 38.57 ± 3.39 | 66.90 ± 3.78 | 68.14 ± 4.19* |
| example 3 | | | |
| Comparative | 35.12 ± 2.04* | 67.49 ± 1.23 | 63.76 ± 2.78** |
| example 4 | | | |
| Comparative | 37.06 ± 1.06 | 64.72 ± 3.00* | 66.09 ± 3.39* |
| example 5 | | | |

Note:
^{###}meant p < 0.001 compared to the blank group;
^{##}meant p < 0.01 compared to the blank group;
***meant p < 0.001 compared to the model group;
**meant p < 0.01 compared to the model group; and
*meant p < 0.05 compared to the model group.

### 4. Inflammatory Hearing Loss

The lipopolysaccharide (LPS) was used to induce an animal model of otitis media. After SD rats were adaptively reared for 7 days and the middle ear and inner ear of the rats were checked to confirm that there was no pathological changes, the rats were divided into a blank group, a model group, a positive group (antideaf otic pills) and sample groups (example 5 to example 13, and comparative example 3 to comparative example 5), a total of 15 groups, each with 6 rats. Except for the blank group, the rats in the rest groups were anesthetized with chloral hydrate, the head was fixed, the external ear canal was cleaned with ear microscopy forceps, after an ear endoscopy, the external ear canal was disinfected with 75% alcohol, the tympanic membrane was exposed by an electric otoscope, and the tympanic membrane was puncture at an anterior inferior position under a direct view, 20 μL of a lipopolysaccharide solution (5 mg/mL) was injected into the tympanic cavity with an insulin syringe for 7 consecutive days. During the modeling period, the rats in the positive group and the sample groups were gavaged with a dosage of 100 mg/kg of rats (0.3 mL/100 g rat body weight per day), and the rats in the blank group and the model group were given gavaged with the same amount of normal saline. At the end of the experiment (the 7^{th} day), the ABR value of each group of rats was detected by the ABR instrument under a condition of 8 kHZ, after the detection, the cochlear tissues of the rats in each group were taken, and the contents of the MDA, the glutamic acid and IL-4 in the cochlear tissues were detected. The results were shown in Table 4. Compared with the blank group, the ABR value, the MDA, the glutamic acid and the IL-4 of the rats in the model group were significantly different (p<0.001, p<0.01), indicating that the model was successful; the ABR value (p<0.05/p<0.01), and/or the MDA (p<0.05), and/or the glutamic acid (p<0.05/p<0.01), and/or the IL-4 (p<0.05/p<0.01) of the rats in the example 5 and 10 to 13 groups were significantly different through the regulation, the effect was good, and there was no disclosure of use of rhizoma curcumae longae in relieving inflammatory hearing loss in the prior art; the ABR value (p<0.001), the MDA (p<0.001), the glutamic acid (p<0.001) and the IL-4 (p<0.001) of the rats in example 6 to 9 groups were significantly different through regulation, and the effect was good and significantly better than those of the rats in comparative examples 3 to 5; the ABR value (p<0.001), the glutamic acid (p<0.001) and the MDA (p<0.001) of the rats in the positive group were significantly different, but the IL-4 showed no significant difference, indicating the compositions of examples 6 to 9 had a significant better effect than that of the positive group; especially, the reduction value of the ABR of the rats in the example 6 to 9 groups (the composition was prepared from rhizoma curcumae longae, fructus Piperis longi, Dendrobium officinale, ginseng, mulberry, Radix puerariae, Cornus officinalis, Semen ziziphi spinosae, Radix astragali, peach kernel, licorice, and honeysuckle) (16.5, 16.5, 13.22 and 15.75 dB respectively) relative to the model group was all higher than the sum of that of the rats in example 5 (the composition was prepared from rhizoma curcumae longae, fructus Piperis longi and Dendrobium officinale) relative to the model group and that of the rats in comparative example 3 (the composition was prepared from ginseng, mulberry, Radix puerariae, Cornus officinalis, Semen ziziphi spinosae, Radix astragali, peach kernel, licorice and honeysuckle) to the model group (6.16+2.33=8.49 dB); the reduction value of the GLU of the rats in the example 6 to 9 groups (the composition was prepared from rhizoma curcumae longae, fructus Piperis longi, Dendrobium officinale, ginseng, mulberry, Radix puerariae, Cornus officinalis, Semen ziziphi spinosae, Radix astragali, peach kernel, licorice, and honeysuckle) (13.84, 19.08, 17.88 and 16.74 mg/L respectively) relative to the model group was all higher than the sum of that of the rats in example 5 (the composition was prepared from rhizoma curcumae longae, fructus Piperis longi and Dendrobium officinale) relative to the model group and that of the rats in comparative example 3 (the composition was prepared from ginseng, mulberry, Radix puerariae, Cornus officinalis, Semen ziziphi spinosae, Radix astragali, peach kernel, licorice and honeysuckle) to the model group (6.98+3.9=10.88 mg/L); the reduction value of the IL-4 of the rats in the example 6 to 9 groups (the composition was prepared from rhizoma curcumae longae, fructus Piperis longi, Dendrobium officinale, ginseng, mulberry, Radix puerariae, Cornus officinalis, Semen ziziphi spinosae, Radix astragali, peach kernel, licorice, and honeysuckle) (130.12, 166.45, 209.93 and 156.53 pg/mL respectively) relative to the model group was all higher than the sum of that of the rats in example 5 (the composition was prepared from rhizoma curcumae longae, fructus Piperis longi and Dendrobium officinale) relative to the model group and that of the rats in comparative example 3 (the composition was prepared from ginseng, mulberry, Radix puerariae, Cornus officinalis, Semen ziziphi spinosae, Radix astragali, peach kernel, licorice and honeysuckle) to the model group (38.67+51.36=90.03 pg/mL); thus the compositions of examples 6 to 9 had synergistic effects in reducing the ABR, GLU and IL-4.

TABLE 4
Experimental results of inflammatory hearing loss of rats

| Experimental group | ABRvalue (dB) | MDA(U/mL) | GLU(mg/L) | IL-4 (pg/mL) |
|---|---|---|---|---|
| Blank group | 41.50 ± 3.27 | 50.33 ± 2.19 | 76.67 ± 2.15 | 222.03 ± 29.01 |
| Model group | 57.83 ± 3.68^{##} | 68.47 ± 3.21^{##} | 92.27 ± 2.34^{###} | 361.92 ± 27.91^{###} |
| Positive group | 40.43 ± 4.72*** | 56.56 ± 3.02** | 75.03 ± 1.79*** | 324.27 ± 11.17 |
| Example 5 group | 51.67 ± 2.19* | 57.14 ± 3.77* | 85.29 ± 1.98* | 323.25 ± 35.89 |
| Example 6 group | 41.33 ± 3.15*** | 56.83 ± 2.76** | 78.43 ± 3.00*** | 231.80 ± 22.71*** |
| Example 7 group | 41.33 ± 2.25*** | 56.21 ± 2.21** | 73.19 ± 2.31*** | 195.47 ± 29.58*** |
| Example 8 group | 44.61 ± 3.98*** | 52.76 ± 2.02** | 74.39 ± 3.24*** | 151.99 ± 15.17*** |
| Example 9 group | 42.08 ± 2.21*** | 54.21 ± 3.16** | 75.53 ± 2.53*** | 205.39 ± 14.38*** |
| Example 10 group | 50.16 ± 4.91 | 59.09 ± 4.01* | 82.88 ± 3.00* | 300.98 ± 18.15* |
| Example 11 group | 47.47 ± 2.55** | 57.75 ± 3.25* | 81.05 ± 2.19** | 263.73 ± 22.77** |
| Example 12 group | 53.11 ± 1.39 | 59.15 ± 2.47* | 85.26 ± 2.58* | 277.35 ± 19.43** |
| Example 13 group | 46.61 ± 3.00** | 57.00 ± 4.64* | 80.37 ± 3.55** | 254.80 ± 37.46** |
| Comparative | 55.50 ± 4.64 | 61.12 ± 3.42* | 88.37 ± 4.23* | 310.56 ± 27.26 |
| example 3 group | | | | |
| Comparative | 51.22 ± 2.23* | 61.18 ± 1.47* | 84.52 ± 4.11* | 297.21 ± 20.71* |
| example 4 group | | | | |
| Comparative | 52.58 ± 1.09* | 57.98 ± 2.06* | 85.09 ± 2.25* | 283.72 ± 30.61* |
| example 5 group | | | | |

Note:
^{###}meant p < 0.001 compared to the blank group;
^{##}meant p < 0.01 compared to the blank group;
***meant p < 0.001 compared to the model group;
**meant p < 0.01 compared to the model group; and
*meant p < 0.05 compared to the model group.

The above experimental results indicated that the Chinese herbal medicine extract/Chinese herbal composition for relieving hearing loss provided by the present disclosure had significant effects in relieving hearing loss such as aging hearing loss, noise-induced hearing loss, and inflammatory hearing loss. Especially, the compositions of examples 6 to 9 had better relieving effects (mean value or significance of the indexes) than those of the positive group and the comparative example groups. In particular, the compositions of examples 6 to 9 had better effects in improving the indexes of aging hearing loss, noise-induced hearing loss, and inflammatory hearing loss (ABR value and/or MDA and/or GLU and/or IL-4) relative to the model group than the sum of the effects of the composition of example 5 (the composition was prepared from rhizoma curcumae longae, fructus Piperis longi and Dendrobium officinale) relative to the model group and the composition of comparative example 3 (the composition was prepared from ginseng, mulberry, Radix puerariae, Cornus officinalis, Semen ziziphi spinosae, Radix astragali, peach kernel, licorice and honeysuckle) relative to the effect of the model group, indicating that the compositions of examples 6 to 9 had synergistic effects in improving the indexes of aging hearing loss, noise-induced hearing loss, and inflammatory hearing loss (ABR value and/or MDA and/or GLU and/or IL-4).

### 5. Taste

Sensory evaluation was performed on the Chinese herbal medicine extracts/Chinese herbal medicine compositions obtained in examples 5 to 13 and comparative examples 3 to 5 (a total of 30 sensory evaluation testers were included after qualified training). 0.5 g of the compositions of examples 5 to 13 and comparative examples 3 to 5 was separately added to 100 mL of water to be prepared into a 0.5% aqueous dispersion without containing any flavoring agents or auxiliary materials, and bitter and pungent tastes were tested. A rhizoma curcumae longae extract was obtained by a 60% ethanol water extraction process (Chinese herbal medicine extract in example 1) as a reference for a pungent control group. The specific results were shown in Table 5: the compositions obtained in examples 6 to 9 only had a little bitter and pungent tastes, thus the prescription combinations in examples 6 to 9 helped to improve the taste of the prescription extract powder.

TABLE 5
Taste test of compositions obtained
in examples and comparative examples

| Experimental group | Bitter taste | Pungent taste |
|---|---|---|
| Pungent control group | − | + + + + |
| Example 5 group | + | + + + |
| Example 6 group | + | − |
| Example 7 group | + | + |
| Example 8 group | + | − |
| Example 9 group | + | − |
| Example 10 group | + + + | + + |
| Example 11 group | + + + | + + + |
| Example 12 group | + + | + + |
| Example 13 group | + + | + + |
| Comparative | + + + | − |
| example 3 | | |
| Comparative | + + + | + + |
| example 4 | | |
| Comparative | + + | − |
| example 5 | | |

Note:
the mark ‘−’ meant that there was no obvious bitter or pungent taste;
‘+’ meant a little bitter or pungent taste;
‘+ + +’ meant relatively obvious bitter or pungent taste; and
‘+ + + +’ meant obvious bitter or pungent taste.

The examples are preferred embodiments of the present disclosure, but the embodiments of the present disclosure are not limited by the examples, and any other changes, modifications, substitutions, combinations and simplification made without departing from the spirit and principle of the present disclosure, all should be equivalent replacement methods and included in the protection scope of the present disclosure.

What is claimed is:

1. A product comprising rhizoma curcumae longae, wherein the product has any one of functions from (1) to (6):
(1) preventing and/or treating hearing loss;
(2) reducing an auditory brainstem response (ABR) value;
(3) reducing the content of malondialdehyde;
(4) reducing the content of glutamic acid;
(5) reducing the content of IL-4; and
(6) inhibiting production of inflammatory factors.

2. A Chinese herbal medicine composition prepared from the raw materials comprising any one of (7) to (10):
(7) 35-85 parts by weight of rhizoma curcumae longae and 35-85 parts by weight of fructus Piperis longi;
(8) 35-85 parts by weight of rhizoma curcumae longae and 25-65 parts by weight of Dendrobium nobile;
(9) 35-85 parts by weight of rhizoma curcumae longae, 25-65 parts by weight of Dendrobium nobile and 35-85 parts by weight of fructus Piperis longi; and
(10) 25-65 parts by weight of Dendrobium nobile and 35-85 parts by weight of fructus Piperis longi.

3. The Chinese herbal medicine composition according to claim 2, prepared from the raw materials comprising any one of (11) to (15):
(11) 35-85 parts by weight of rhizoma curcumae longae, 35-85 parts by weight of fructus Piperis longi, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle;
(12) 35-85 parts by weight of rhizoma curcumae longae, 25-65 parts by weight of Dendrobium nobile, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle;
(13) 35-85 parts by weight of rhizoma curcumae longae, 25-65 parts by weight of Dendrobium nobile, 35-85 parts by weight of fructus Piperis longi, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle;
(14) 25-65 parts by weight of Dendrobium nobile, 35-85 parts by weight of fructus Piperis longi, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle; and
(15) 35-85 parts by weight of rhizoma curcumae longae, 15-65 parts by weight of ginseng, 15-65 parts by weight of mulberry, 5-45 parts by weight of Radix puerariae, 5-45 parts by weight of Cornus officinalis, 5-45 parts by weight of Semen ziziphi spinosae, 5-45 parts by weight of Radix astragali, 5-45 parts by weight of peach kernel, 3-22 parts by weight of licorice, and 3-22 parts by weight of honeysuckle.

4. A preparation method of the Chinese herbal medicine composition according to claim 2, comprising:
conducting water extraction and/or alcohol extraction of any one of (7) to (10) as a raw material to obtain the Chinese herbal medicine composition.

5. A preparation method of the Chinese herbal medicine composition according to claim 3, comprising:
conducting water extraction and/or alcohol extraction of any one of (11) to (15) as a raw material to obtain the Chinese herbal medicine composition.

6. A product comprising the Chinese herbal medicine composition according to claim 2, wherein the product has any one of functions from (1) to (6):
(1) preventing and/or treating hearing loss;
(2) reducing an auditory brainstem response (ABR) value;
(3) reducing the content of malondialdehyde;
(4) reducing the content of glutamic acid;
(5) reducing the content of IL-4; and
(6) inhibiting production of inflammatory factors.

7. The product according to claim 6, wherein the type of the product comprises a food and a medicine.

8. A product comprising the Chinese herbal medicine composition according to claim 3, wherein the product has any one of functions from (1) to (6):
(1) preventing and/or treating hearing loss;
(2) reducing an auditory brainstem response (ABR) value;
(3) reducing the content of malondialdehyde;
(4) reducing the content of glutamic acid;
(5) reducing the content of IL-4; and
(6) inhibiting production of inflammatory factors.

9. A product comprising the Chinese herbal medicine composition according to claim 2.

10. The product according to claim 9, wherein the type of the product comprises a food and a medicine.

11. The product according to claim 10, wherein the type of the food may comprise a beverage, a biscuit, a candy and a pastry.

12. The product according to claim 10, wherein a dosage form of the medicine comprises a capsule, a tablet, a granule, a mixture, a decoction, a pill, an electuary, a dripping pill, a liniment, a tincture and a wine.
